(19) United States
(12) Patent Application Publication
(10) Pub. No.: US 2010/0047742 A1
(43) Pub. Date: Feb. 25, 2010

(54) TUBULE-BLOCKING SILICA MATERIALS FOR DENTIFRICES

(76) Inventors: William Henry Pitcock, JR., Havre de Grace, MD (US); Karl W. Gallis, Perryville, MD (US); John V. Offidani, Havre de Grace, MD (US); Michael S. Darsillo, Landenberg, PA (US)

Correspondence Address: J M HUBER CORPORATION, 1000 Parkwood Circle, Ste. 1000, PATENT DEPARTMENT, Atlanta, GA, 30339, US

(21) Appl. No.: 12/499,359

(22) Filed: Jul. 8, 2009

Related U.S. Application Data
(60) Provisional application No. 61/196,732, filed on Aug. 25, 2008.

(51) Int. Cl. A61C 5/08; A61K 8/25

(52) U.S. Cl. 433/215; 424/401

(57) ABSTRACT

Precipitated silica materials are provided for utilization as abrasives or thickeners within dentifrice formulations that simultaneously effectuate tubule blocking within tooth dentin to reduce dentin sensitivity. Such precipitated silica materials have sufficiently small particle sizes and exhibit certain ionic charge levels by, for example, adjusting the zeta potential properties of the precipitated silica materials through treatment with certain metals to permit effective static attraction and eventual accumulation within dentin tubules when applied to teeth from a dentifrice formulation.

### CROSS-REFERENCE TO RELATED APPLICATIONS

The present application claims the benefit of priority to U.S. Provisional Application Ser. No. 61/196,732, filed Aug. 25, 2008, entitled “Tubule-Blocking Silica Materials for Dentifrices”, the disclosure of which is hereby incorporated by reference in its entirety.

### TECHNICAL FIELD OF THE INVENTION

This invention pertains to precipitated silica materials for utilization as abrasives or thickeners within dentifrice formulations, and more particularly to such precipitated silica materials that simultaneously effectuate tubule blocking within tooth dentin.

### BACKGROUND OF THE INVENTION

Silica materials are particularly useful in dentifrice products, such as toothpastes, where they function as abrasives and thickeners. In addition to this functional versatility, silica materials, particularly amorphous precipitated silica materials, also have the advantage, when compared to other dentifrice abrasives such as alumina and calcium carbonate, of having a relatively high compatibility with active ingredients like fluoride sources including sodium fluoride, sodium monofluorophosphate, etc. Particularly relevant to their use in dentifrices is that such silica materials offer simultaneously good cleaning properties and moderate dentin abrasion levels in order to accord the user a dentifrice that effectively cleans tooth surfaces without detrimentally abrading such surfaces. The ability to provide a fluoride-compatible thickening agent for toothpaste formulations is also of great benefit to the consumer and manufacturer alike.

Tooth sensitivity has become an issue recently within the dentifrice arena, particularly in terms of the loss of enamel protection due to different eating habits and dental cleaning rituals of certain people. As such, in addition to the aforementioned abrasive and thickening benefits imparted to dentifrice products by silica materials, formulators of certain specialty dentifrice products have taken to incorporating certain materials that are useful for reducing tooth sensitivity to certain degrees. In particular, toothpastes have been designed to reduce the sensitivity of teeth to hot and cold temperatures and additional active stimuli like polysaccharide sweets and thus reduce the pain and/or discomfort associated with such undesirable sensations.

Although the causes of teeth sensitivity are not known with certainty, it is believed that sensitivity is related to exposed dentinal tubules. These tubules, which contain fluid and cellular structures, extend outward from the tooth pulp, to the surface or border of the enamel. According to some theories, age, lack of proper dental hygiene, and/or medical conditions can result in enamel loss or gum recession on the surface of teeth. Depending upon the severity of the enamel loss or gum recession, the outer portions of the dentinal tubules may become exposed to the external environment of the mouth. When these exposed tubules come into contact with certain stimuli, such as, for example, hot or cold liquids, the dentinal fluid may expand or contract causing pressure differentials within the teeth that results in discomfort and possibly pain to the subject person.

Prior efforts to address such increased sensitivity have focused on disrupting the potassium/sodium ion channel pump responsible for sending pain sensation to the brain. It is generally believed, without intending to depend on any specific scientific theory, that such a chemical mechanism has historically been imparted to a user through the inclusion of potassium nitrate within a dentifrice formulation. This alternative merely, however, prevents the ability of the body to send pain sensations; the pain still occurs, but is not actually felt by the user. This illusory effect is temporary and is lost with time, thereby requiring continued utilization of potassium nitrate-containing toothpaste for effect maintenance. Other efforts at reducing sensitivity have centered on occluding tubules within exposed dentin. In such a manner, tubule occlusion is achieved through the covering or filling of the tubule with a material such as certain types of silica materials. In preparing this “occluding material,” however, the focus has typically concerned controlling particle size to be of a size to at least partially cover the tubule opening. However, in most cases selecting occluding material based on particle size is not by itself sufficient to provide enough occlusion to obtain satisfactory sensitivity-blocking performance. Generally, the occluding material will not exhibit an affinity for the tooth surface and will thus lack proper adhesive capability to retain within, on, or around the subject tubule for a sufficient period of time to reduce the sensitivity level thereof to the necessary degree for sufficient pain and/or discomfort control, prevention, or otherwise reduction. For instance, standard precipitated silica materials will possibly occlude on a temporary basis (if provided at a suitably small particle size for such occlusion within a target tubule), but are easily removed when, for instance, the user rinses his or her mouth out with water after brushing. There is thus a need in the art for a new silica material that exhibits proper fluoride compatibility (at least with some fluoride sources), effectively small particle size for proper introduction within target dentinal tubules, proper static charge for long-term stability when introduced within a dentinal tubule, and the capability to be transferred in such a manner to a tooth and within a dentinal tubule during introduction into a user's mouth cavity and contact with the subject tooth surface during typical tooth brushing. To date, no such silica material has been provided that provides such beneficial results.

### BRIEF SUMMARY OF THE INVENTION

A significant advantage of the present embodiments is the sufficient degree of affinity with target dentin surfaces exhibited by the adduct-treated precipitated silica materials to permit long-term adhesion on such dentin surfaces allowing for entry and filling of tubules therein. Another advantage of the embodiments is the ability to include such adduct-treated precipitated silica materials in dentifrice formulations as either abrasives or thickening agents and, upon brushing of the subject's teeth, such adduct-treated precipitated silica materials will transfer from the dentifrice to the tooth surfaces and occlude the target dentinal tubules.

Accordingly, in one embodiment a dentifrice comprises a precipitated silica material having a mean particle size of 1 to 5 microns and having an adduct present on at least a portion of its surface to form an adduct-treated precipitated silica material, wherein the adduct-treated precipitated silica material exhibits a zeta potential of greater than 10% of the zeta potential of a precipitated silica material of the same structure on which no adduct is present. Also encompassed is a dentifrice comprising such adduct-treated precipitated silica materials as a thickening agent, abrasive agent, or both and comprising at least one other component such as a solvent, a preservative, a surfactant, or an abrasive or thickening agent other than the adduct-treated precipitated silica materials.

Also encompassed is a method of treating a mammalian tooth comprising the steps of

a) providing a dentifrice comprising a precipitated silica material having a mean particle size of 1 to 5 microns and having an adduct present on at least a portion of its surface to form an adduct-treated precipitated silica material that exhibits a zeta potential reduction greater than 10% when compared to a precipitated silica material of the same structure on which no adduct is present;

b) applying the dentifrice to a mammalian tooth; and

c) brushing the dentifrice-applied tooth of step “b” thereby permitting occlusion of subject dentinal tubules with the adduct-treated precipitated silica material.

### BRIEF DESCRIPTION OF THE DRAWINGS

FIG. 1 is a series of photomicrographs showing the results of the dentifrice affinity test of a Control sample in terms of occlusion capability within dentinal tubules.

FIG. 2 is a series of photomicrographs showing the results of the dentifrice affinity test of Comparative 1 in terms of occlusion capability within dentinal tubules.

FIG. 3 is a series of photomicrographs showing the results of the dentifrice affinity test of Example 6 in terms of occlusion capability within dentinal tubules.

FIG. 4 is a series of photomicrographs showing the results of the dentifrice affinity test of Comparative 4 in terms of occlusion capability within dentinal tubules.

FIG. 5 is a series of photomicrographs showing the results of the dentifrice affinity test of Comparative 5 in terms of occlusion capability within dentinal tubules.

FIG. 6 is a series of photomicrographs showing the results of the dentifrice affinity test of Comparative 2 in terms of occlusion capability within dentinal tubules.

### DETAILED DESCRIPTION OF THE INVENTION

All parts, percentages and ratios used herein are expressed by weight unless otherwise specified. All documents cited herein are incorporated by reference.

Precipitated silica materials for use in dentifrice compositions have been developed with increased affinity towards a mammalian tooth particle, thus adhering strongly to the tooth surface and providing greater occlusion over the dentinal tubules. Without being limited by theory it is believed that the increased affinity between the precipitated silica material and teeth is a consequence of the reduction of the negative charge on the surface of the precipitated silica material; this reduction is accomplished by the presence of an adduct on at least a portion of the surface of the silica.

The surface charge of silica, and manipulating that surface charge, is a much studied and explored area, if also somewhat contentious. (See e.g., Ralph K. Iler, The Chemistry of Silica: Solubility, Polymerization, Colloid and Surface Properties and Biochemistry of Silica, pp. 659-672). The use of some adducts has also been previously discussed in the patent literature, e.g., Wason, U.S. Pat. No. 3,967,563, and Wason, U.S. Pat. No. 4,122,160, although such silica materials were treated with metal adducts solely for the ability to generate transparent abrasives exhibiting large particle sizes for dentifrices.

Accordingly, in a certain embodiment a precipitated silica material has a mean particle size of 1 to 5 microns and has an adduct present on at least a portion of its surface to form an adduct-treated precipitated silica material, wherein the adduct-treated precipitated silica material exhibits a zeta potential reduction greater than 10% when compared to a precipitated silica material of the same structure on which no adduct compound is present.

In one embodiment, the adduct is a metal element. In another embodiment, the adduct is a metal element selected from the transition metals and post-transition metals. Examples of suitable metal elements include aluminum, zinc, tin, strontium, iron, copper, and mixtures thereof. The adduct-treated precipitated silica material is formed by the addition of the adduct in the form of a water-soluble metal salt during the formation of precipitated silica material. Any metal salt that is soluble in acidic conditions would be suitable, such as metal nitrates, metal chlorides, metal sulfates, and the like.

In one embodiment, the adduct-treated precipitated silica material exhibits a zeta potential reduction greater than 15% when compared to a precipitated silica material of the same structure on which no adduct is present. In another embodiment, the zeta potential reduction is greater than 20%. In still another embodiment, the zeta potential reduction is greater than 25%.

In one embodiment, the adduct-treated precipitated silica material is prepared according to the following process. An aqueous solution of an alkali silicate, such as sodium silicate, is charged into a reactor equipped with mixing means adequate to ensure a homogeneous mixture. The alkali silicate solution in the reactor is preheated to a temperature of between about 65° C. and about 100° C. The alkali silicate solution may have an alkali silicate concentration of approximately 8.0 to 35 wt %, such as from about 8.0 to about 20 wt %. The alkali silicate may be a sodium silicate with a SiO₂:Na₂O ratio of from about 1 to about 3.5, such as about 2.4 to about 3.4. The quantity of alkali silicate charged into the reactor is about 5 wt % to 100 wt % of the total silicate used in the batch. Optionally, an electrolyte, such as sodium sulfate solution, may be added to the reaction medium. Additionally, this mixing may be performed under high-shear conditions.

To the reactor is then simultaneously added: (1) an aqueous solution of an acidulating agent or acid, such as sulfuric acid; (2) additional amounts of an aqueous solution containing the same species of alkali silicate as is in the reactor, such aqueous solution being preheated to a temperature of about 65° C. to about 100° C. An adduct compound is added to the acidulating agent solution prior to the introduction of the acidulating agent solution into the reactor. The adduct compound is premixed with the acidulating agent solution in a concentration of mol. of adduct compound to L of acidulating agent solution of about 0.002 to about 0.185, preferably about 0.074 to about 0.150. Optionally, if higher adduct concentrations are required in the adduct-treated precipitated silica material, an aqueous solution of the adduct compound can be used in place of the acid.

The acidulating agent solution preferably has a concentration of acidulating agent of about 6 to 35 wt %, such as about 9.0 to about 20 wt %. After a period of time the inflow of the alkali silicate solution is stopped and the acidulating agent solution is allowed to flow until the desired pH is reached.

The reactor batch is allowed to age or “digest” for between 5 minutes to 30 minutes at a set digestion temperature, with the reactor batch being maintained at a constant pH. After the completion of digestion, the reaction batch is filtered and washed with water to remove excess by-product inorganic salts until the wash water from the silica filter cake obtains a conductivity of less than about 2000 μ mhos. Because the conductivity of the silica filtrate is proportional to the inorganic salt by-product concentration in the filter cake, then by maintaining the conductivity of the filtrate to be less than 2000 μ mhos, the desired low concentration of inorganic salts, such as Na₂SO₄ in the filter cake may be obtained. The silica filter cake is slurried in water, and then dried by any conventional drying techniques, such as spray drying, to produce adduct-treated precipitated silica material containing from about 3 wt % to about 50 wt % of moisture. The adduct-treated precipitated silica material may then be milled to obtain the desired particle size of between about 1 μ m to 5 μ m. Such a particle size is imperative to provide the beneficial abrasive and/or thickening properties when in the target dentifrice formulation as well as impart the desired occlusion of dentinal tubules to reduce pain and discomfort as noted above for the subject person.

For purposes herein, a “dentifrice” has the meaning defined in Oral Hygiene Products and Practice, Morlon Pader, Consumer Science and Technology Series, Vol. 6, Marcel Dekker, NY 1988, p. 200, which is incorporated herein by reference. Namely, a “dentifrice” is “ . . . a substance used with a toothbrush to clean the accessible surfaces of the teeth. Dentifrices are primarily composed of water, detergent, humectant, binder, flavoring agents, and a finely powdered abrasive as the principal ingredient . . . a dentifrice is considered to be an abrasive-containing dosage form for delivering anti-caries agents to the teeth.” Dentifrice formulations contain ingredients which must be dissolved prior to incorporation into the dentifrice formulation (e.g. anti-caries agents such as sodium fluoride, sodium phosphates, flavoring agents such as saccharin).

When incorporated within a dentifrice formulation, the adduct-treated precipitated silica material may be present in an amount of from 0.01 to about 25% of the total weight of the entire dentifrice itself. If the adduct-treated precipitated silica material is abrasive in nature, the amount may be from 0.05 to about 15% by weight (the abrasive may act alone, or as a booster type that simultaneously provides tubule occlusion after brushing is performed). If the adduct-treated precipitated silica material is a viscosity modifiers (thickening agents), the amount may be from 0.05 to about 10% by weight. The adduct-treated precipitated silica material with the proper metal adduct present thereon for zeta potential modifications will simultaneously provide both viscosity modification and long-term tubule occlusion. If needed, however, the adduct-treated precipitated silica material does not necessarily require any characteristic other than as a tubule occluding material. As such, the amount may be within the range noted above within the dentifrice formulation, but the materials will not provide any appreciable degree of thickening or abrasivity to the dentifrice, but solely will provide tubule occlusion benefits. Such formulations may also include potassium nitrate salts, as one example, of a suitable other desensitizing materials, if desired.

The compositions and methods described above will be further understood with reference to the following non-limiting examples.

### EXAMPLES

Examples were prepared to study the effect on the affinity of the silica for a mammalian tooth by adding an adduct to precipitated silica materials. In the first set of batches, prepared at pilot plant scale, several samples were prepared containing the metal adduct Al₂O₃, while one comparative sample used had only trace amounts of aluminum or other metals as shown in Table 1. The samples, below, were prepared as follows:

The quantities of reactants and the reactant conditions are set forth in Table 1, below. First, 67 L of an aqueous solution containing 19.5 wt % of sodium silicate (having a 3.32 molar ratio of SiO₂:Na₂O) and 167 L of water was charged into a 400 gallon reactor heated to 87° C. with recirculation at 30 Hz and stirring at 60 RPM. An aqueous solution of sulfuric acid (having a concentration of 17.1 wt % and containing aluminum in the concentration per acid solution specified in Table 1, below) and an aqueous solution of sodium silicate (at a concentration of 19.5 wt %, the sodium silicate having a 3.32 mole ratio, the solution heated to 85° C.) were then added simultaneously at rates of 12.8 L/min (for silicate) and 1.2 L/min (for sulfuric acid) for 47 minutes. After 47 minutes the silicate addition was stopped and the acid addition continued until the reactor batch pH dropped to 5.5. The batch temperature was then maintained at 87° C. for ten minutes to allow the batch to digest. The silica batch was then filtered and washed to form a filter cake having a conductivity of about 1500 μ mhos. The filter cake was then slurried with water, spray dried, and the spray dried product micronized by a suitable technique including jet-milling or air-milling to a particle size of about 3 μ m. A comparative precipitated silica (Comparative 2) was prepared by hammer-milling the material of Example 6 to an average particle size of approximately 10 μ m. The materials were then tested for the presence of several different metal oxides, with the concentrations listed below in Table 1.

TABLE 1
Metal Adduct Additions

| | Mol | | | | | | |
|---|---|---|---|---|---|---|---|
| | Al/L of | | | | | | |
| | acid | Al₂O₃ | CaO | Fe₂O₃ | MgO | Na₂O | TiO₂ |
| Sample ID | solution | (ppm) | (ppm) | (ppm) | (ppm) | (ppm) | (ppm) |
| Comparative 1 | — | 771 | 26 | 157 | 60 | 1.29 | 135 |
| Example 1 | 0.007 | 1100 | 31 | 159 | 68 | 1.15 | 137 |
| Example 2 | 0.014 | 1500 | 38 | 150 | 72 | 0.96 | 139 |
| Example 3 | 0.028 | 3900 | 30 | 144 | 74 | 1.03 | 137 |
| Example 4 | 0.055 | 7300 | 40 | 144 | 77 | 1.70 | 133 |
| Example 5 | 0.110 | 15400 | 44 | 143 | 89 | 1.29 | 133 |
| Example 6 | 0.220 | 19600 | 37 | 141 | 79 | 1.48 | 131 |
| Comparative 2 | 0.220 | 19600 | 37 | 141 | 79 | 1.48 | 131 |

### Analysis of Inventive Materials for Tubule Occlusion and Other Characteristics

The various silica materials described herein were measured as follows, unless indicated otherwise.

The CTAB external surface area of silica was determined by adsorption of CTAB (cetyltrimethylammonium bromide) on the silica surface, the excess separated by centrifugation and determined by titration with sodium lauryl sulfate using a surfactant electrode. The external surface of the silica was determined from the quantity of CTAB adsorbed (analysis of CTAB before and after adsorption).

Specifically, about 0.5 g of silica was accurately weighed and placed in a 250-ml beaker with 100.00 ml CTAB solution (5.5 g/L, adjusted to pH 9.0±0.2), mixed on an electric stir plate for 30 minutes, then centrifuged for 15 minutes at 10,000 rpm. 1.0 ml of 10% Triton X-100 is added to 5.0 ml of the clear supernatant in a 100-ml beaker. The pH was adjusted to 3.0-3.5 with 0.1 N HCl and the specimen was titrated with 0.0100 M sodium lauryl sulfate using a surfactant electrode (Brinkmann SUR15O1-DL) to determine the endpoint.

The oil absorption values were measured using the rubout method. This method is based on a principle of mixing linseed oil with a silica by rubbing with a spatula on a smooth surface until a stiff putty-like paste is formed. By measuring the quantity of oil required to have a paste mixture which will curl when spread out, one can calculate the oil absorption value of the silica—the value which represents the volume of oil required per unit weight of silica to saturate the silica sorptive capacity. A higher oil absorption level indicates a higher structure of precipitated silica; similarly, a low value is indicative of what is considered a low-structure precipitated silica. Calculation of the oil absorption value was done as follows:

$$Oil absorption = \frac{ml oil absorbed}{weight of silica , grams} \times 100 \\ = ml oil / 100 gram silica$$

Median particle size was determined using a Model LA-930 (or LA-300 or an equivalent) laser light scattering instrument available from Horiba Instruments, Boothwyn, Pa.

The % 325 mesh residue of silica was measured utilizing a U.S. Standard Sieve No. 325, with 44 micron or 0.0017 inch openings (stainless steel wire cloth) by weighing a 10.0 gram sample to the nearest 0.1 gram into the cup of a 1 quart Hamilton mixer Model No. 30, adding approximately 170 ml of distilled or deionized water and stirring the slurry for at least 7 min. The mixture was transferred onto the 325 mesh screen and water was sprayed directly onto the screen at a pressure of 20 psi for two minutes, with the spray head held about four to six inches distant from the screen. The remaining residue was then transferred to a watch glass and dried in an oven at 150° C. for approx. 15 min.; then cooled and weighed on an analytical balance.

The pH values of the reaction mixtures (5 weight % slurry) can be monitored by any conventional p1H sensitive electrode.

To measure brightness, samples were pressed into a smooth surfaced pellet and evaluated with a Technidyne Brightmeter S-5/BC. This instrument has a dual beam optical system where the sample is illuminated at an angle of 45°, and the reflected light viewed at 0°.

For the materials produced above, measurements of these properties were undertaken and provided in Table 2.

TABLE 2
Properties of Prepared Precipitated Silica Materials

| | | | | | Mean | Median | | | |
|---|---|---|---|---|---|---|---|---|---|
| | | 325 | | | Particle | Particle | Oil | | |
| | H₂O | residue | BET | CTAB | Size | Size | Absorption | 5% | |
| | (%) | (%) | (m²/g) | (m²/g) | (μm) | (μm) | (cc/100 g) | pH | Brightness |
| Comparative 1 | 6.4 | 0.00 | 185 | 34 | 2.2 | 2.2 | 92 | 7.4 | 98.6 |
| Example 1 | 5.7 | 0.01 | 213 | 31 | 2.5 | 2.5 | 91 | 7.7 | 98.2 |
| Example 2 | 5.9 | 0.02 | 212 | 46 | 2.7 | 2.2 | 99 | 7.9 | 98.9 |
| Example 3 | 6.1 | 0.00 | 210 | 48 | 2.4 | 2.4 | 102 | 8.1 | 99.4 |
| Example 4 | 6.3 | 0.00 | 222 | 44 | 2.8 | 2.8 | 91 | 7.7 | 99.6 |
| Example 5 | 6.2 | 0.00 | 315 | 53 | 3.1 | 3.0 | 91 | 8.4 | 98.9 |
| Example 6 | 5.7 | 0.00 | 349 | 68 | 3.6 | 3.5 | 89 | 8.2 | 99.0 |
| Comparative 2 | 5.7 | 0.10 | 349 | 68 | 10.9 | 9.5 | 89 | 8.2 | 99.0 |

Zeta potential is a measure of the charge on the external surface of a particle suspended in solution. Particles with zeta potentials of the same charge will tend to repel one another and particles with zeta potentials of the opposite charge will tend to be attracted to one another. Historically, zeta potential has been determined by microelectrophoresis, whereby an electric field is applied across a dispersion of particles and the velocity of the particles as they migrate toward an electrode of opposite charge is measured. Particles traveling at a greater velocity toward the electrode of opposite charge will tend to have an increased magnitude of charge on their surface. Alternatively, zeta potential can be determined by electrokinetic sonic amplitude (ESA) technique. ESA measures the electrokinetic properties of a particle by an electroacoustic method. A high frequency oscillating electric field is applied to a dispersion of particles. The particles will oscillate with the applied field proportional to the charge on their surface. As the particles move in one direction, the liquid they displace will move in the other. If there are density differences between the particles and the liquid medium, an acoustic wave will be generated at the interface of the electrode and the liquid dispersion as a result of the liquid that is displaced by the moving particles. The acoustic wave generated can then be measured and the intensity of the wave is then related to the magnitude of the zeta potential. Zeta potential is usually measured across a range of pH values, thus giving an indication of how the surface charge of the suspended particles varies as a function of pH (Greenwood, R. “Review of the measurement of zeta potentials in concentration aqueous suspensions using electroacoustics” Advances in Colloid and Interface Science, 2003, 106, 55-81, herein entirely incorporated by reference). The zeta potential of Comparative 1 and Example 1-6 were measured and the results are tabulated below in Table 3. As can be seen from the Table 3, the negative charge (as measured by the zeta potential) on the surface of the silica was lower for Example 6 at dentifrice pHs (i.e., between about 7 to about 9) than for Comparative 1 (the Comparatives and Examples 1-10 were sent to Colloid Measurements LLC Systems for zeta potential analysis by the ESA method).

TABLE 3
Zeta Potentials

| | | % Reduction in Zeta |
|---|---|---|
| | Zeta Potential | Potential vs. |
| Sample | (at pH 8.0) | Comparatives |
| Comparative 1 | −41.5 | n/a |
| Example 1 | −40.4 | 2.65 |
| Example 2 | −38.5 | 7.23 |
| Example 3 | −39.6 | 4.58 |
| Example 4 | −38.4 | 7.47 |
| Example 5 | −34.2 | 17.59 |
| Example 6 | −29.4 | 29.16 |
| Comparative 3 | −55.8 | n/a |
| Example 7 | −38.3 | 31.36 |
| Example 8 | −33.8 | 39.42 |
| Example 9 | −33.1 | 40.68 |
| Example 10 | −44.3 | 20.61 |
| Comparative 4 | −38.2 | n/a |
| Comparative 5 | −37.3 | 2.36 |

It was observed that the presence of the metal adduct had the effect of reducing the quantity of negative charge on the silica surface.

Next, the affinity between the silicas prepared above and bovine teeth (analogous to all mammalian teeth) was measured by using an atomic force microscope to measure adhesion forces. The use of atomic force microscopy (“AFM”) in this context is itself a novel procedure. Since its initial development over twenty years ago (see Binnig, G.; Quate, F. F. Phys. Rev. Lett., 56, 930 (1986)), AFM has been used in a remarkably broad array of technical fields, including such disparate fields as microelectronics (e.g., Douhéret et al., Progress in Photovoltaics: Research and Applications, 15, 713, 2007); chemistry [e.g., S. Manne et al., Science, 251, 183 (1991)] and especially the biological sciences [see especially B. Drake et al., Science 243, 1586 (1989)]. The versatility of AFM techniques are attributable to a number of factors, but among them are the fact that unlike non-optical microscope technologies such as Electron or Transmission Electron Microscopes (“EM” or “TEM”) and Scanning Electron Microscopes (“SEM”), AFMs do not require a vacuum nor special treatment of samples (e.g., sputtering or plating with a conductive layer of material). AFM is also unique in its ability to provide true three-dimensional measurements and imaging.

Sample preparation for the AFM consisted of compressing the silica to be measured into a 1.25 inch tablet using an Angstrom heavy-duty tablet press (40,000 lbs., 3 minutes hold time). The resulting tablet was then mounted onto a 15 mm AFM specimen disc using double sided adhesive tape. The prepared sample was then mounted on the X-Y stage of the AFM either on the magnetic sample holder or on the vacuum chuck directly on the stage.

Bovine teeth were obtained from the Indiana University School of Dentistry packaged in a solution of thymol. Prior to use they were sterilized in an autoclave and then stored in ethanol. Teeth were allowed to dry before any cutting or grinding was performed. AFM tips (DNP type, cantilever A, k=0.58 N/m nom.) were prepared by filing a bovine tooth with a Dremel #191 High-Speed Cutter on a Dremel 400|XPR rotary tool. A single copper filament (Hex-Wix Fine Braid solder wick, #W76-10), was used to place a small drop of epoxy (Elmers Pro Bond Super Fast Epoxy Resin) on the end of the cantilever. A separate piece of copper filament was then used to select an appropriately shaped particle of tooth (approximately spherical, roughly ˜20-30 μm in diameter) and place it into the epoxy. The AFM tip was then allowed to dry at room temperature overnight.

The AFM tip was mounted in a standard tip holder (Veeco Model #DCHNM, Cantilever Holder) or in a fluid tip holder (Veeco Model #DTFML-DD, Direct Drive Fluid Cantilever Holder) and installed on the scanning probe microscope (SPM) head of the AFM. All measurements were made following the manufacturers instructions and were carried out using a Digital Instruments Dimension 3100 AFM mounted inside an acoustic hood for vibration isolation. The instrument was controlled using NanoScope IIIa version 4.32r3 software. All raw force curve data was exported in units of V, and was converted to obtain the force in nN in a spreadsheet. The conversion was performed using the following equation provided in the Veeco Dimension 3100 users' manual:

Force (nN)=Deflection (V)×Deflection Sensitivity (nm·V⁻¹)×k(nN·nm⁻¹)

where deflection is the deflection measured on the force curve, deflection sensitivity is the slope of deflection versus Z voltage while the tip is in contact with the sample and k is the nominal spring constant of the cantilever.

Measurements were performed in both air and liquid environments. In the case of the liquid environment, a liquid tip holder was used to hold the AFM tip. In order to eliminate variation that may occur from differences in the spring constants of different AFM tips and/or differences in the size and shape of the bovine tooth fragment attached to the AFM tip, the same AFM tip was used for all measurements in a given experiment. Comparative 1 and the silica prepared in Example 6 were evaluated. For simplicity, the adhesion forces for the comparatives were set to 100 percent and the values for the examples were adjusted accordingly. The results are shown in Table 4.

TABLE 4
Adhesion Force Measurements

Adhesion Force

| | In Air | In Liquid |
|---|---|---|
| Comparative 1 | 100 | 100 |
| Example 6 | 219 | 135 |

It was observed that the inventive Example 6 containing the aluminum adduct had a greater adhesion force to the bovine tooth fragment when measured in air and liquid environments.

In order to further verify these results to confirm that these effects are indeed the result of an attractive force between the tooth particle on the cantilever tip and the silica pellet, a study was performed where commercially available AFM tips were used. A sectioned piece of bovine tooth, approximately 1mm×1 mm with the tubule openings oriented approximately 90° to the surface, was used as the substrate. Two different cantilevers, one modified with a 5 μm spherical SiO₂ bead (NovaScan PT.SiO₂.SI.5) and the other modified with a 5 μm spherical Al₂O₃ bead NovaScan PT.CUST.SI), were chosen and affinity measurements were performed. The results of these measurements are shown in Tables 5 and 6. It was observed that the use of the alumina particle resulted in an improvement in affinity over the use of a silica particle in both air and liquid environments. It is noted that different tips were utilized to measure the AFMs for the test subjects in each of Tables 4, 5, and 6 and thus apparent differing results were realized due to the tip differences themselves.

TABLE 5
Adhesion Force Measurements

Adhesion Force

| | In Air | In Liquid |
|---|---|---|
| SiO₂ | 100 | 100 |
| Al₂O₃ | 232 | 285 |

TABLE 6
Adhesion Force in Relation to Metal Adduct Amount

| | | Adhesion Force |
|---|---|---|
| | % Al Adduct | in Air |
| Comparative 1 | 0.077 | 100 |
| Example 1 | 0.110 | 87 |
| Example 2 | 0.150 | 113 |
| Example 3 | 0.390 | 124 |
| Example 4 | 0.730 | 84 |
| Example 5 | 1.540 | 115 |
| Example 6 | 1.960 | 156 |

In order to investigate the effect of the adduct loading level, a study was performed where silica samples were prepared containing increasing levels of adduct. The physical and chemical analysis of these samples is summarized in Tables 1 and 2, and the results of the AFM affinity study are shown in Table 6. It was observed that the Example 6 material exhibited the greatest affinity to the bovine tooth modified AFM tip, and that in general, the addition of the aluminum adduct increased the affinity between the silica and the tooth particle.

In order to investigate the performance of different adducts, a set of samples were prepared according to the following process. 410 mL of silicate (13.3%, 1.112 g/ml, 3.32 MR) were added to the reactor and heated to 85° C. with stirring at 300 RPM. Silicate (13.3%, 1.112 g/ml, 3.32 MR) and sulfuric acid (11.4%, 1.078 g/ml) were then simultaneously added at 82.4 mL/min and 24.8 mL/min for 47 minutes. After 47 minutes, the flow of silicate was stopped and the pH was adjusted to 5.5 with continued flow of acid. Once pH 5.5 was reached, the batch was allowed to digest for 10 minutes at 90° C. After the digestion time was complete, it was filtered, washed with approximately 6 L of deionized water and was dried at 105° C. overnight.

The silica samples were then tested for the presence of several different metal oxides, with the concentrations listed in Table 7. Several other physical properties of these materials were also measured and the results are shown in Table 8.

TABLE 7
Metal Oxide Presence

| | Al₂O₃ | CaO | Fe₂O₃ | MgO | Na₂O | TiO₂ | Cu | Zn | Sn |
|---|---|---|---|---|---|---|---|---|---|
| Sample ID | (ppm) | (ppm) | (ppm) | (ppm) | (ppm) | (ppm) | (%) | (%) | (%) |
| Comparative 3 | 616 | 37 | 245 | 81 | 2.36% | 119 | — | — | — |
| Example 7 | 604 | 131 | 191 | 99 | 1.13% | 117 | 1.39% | — | — |
| Example 8 | 638 | 140 | 198 | 95 | 2.33% | 117 | — | — | 3.63% |
| Example 9 | 646 | 35 | 200 | 70 | 1.17% | 116 | — | 2.78% | — |
| Example 10 | 753 | 58 | 1.95% | 99 | 2.55% | 123 | — | — | — |

TABLE 8
Physical Properties of Different Precipitated Silica Materials

| | | | Oil | |
|---|---|---|---|---|
| | BET | CTAB | Absorption | |
| Sample ID | (m²/g) | (m²/g) | (cc/100 g) | 5% pH |
| Comparative 3 | 60 | 40 | 99 | 9.25 |
| Example 7 | 69 | 48 | 107 | 8.95 |
| Example 8 | 54 | 38 | 93 | 9.40 |
| Example 9 | 53 | 30 | 105 | 8.30 |
| Example 10 | 58 | 47 | 94 | 9.80 |

The samples were pressed into pellets and were analyzed by the previously described AFM method. It was observed that silica materials containing metal adducts exhibited increased adhesion forces than the comparative silica materials prepared without metal adducts (or only trace amounts of adducts). In particular silica materials with 1.4% Cu, 3.6% Sn, and 2.0% Al all exhibited adhesion forces greater than the Comparative 3 silica containing no adducts.

TABLE 9
Adhesion Force Measurements

| | Adduct | Adhesion Force in Air |
|---|---|---|
| Comparative 3 | None | 100 |
| Example 6 | 2.0% Al₂O₃ | 325 |
| Example 7 | 1.4% Cu | 325 |
| Example 8 | 3.6% Sn | 297 |
| Example 9 | 2.8% Zn | 230 |
| Example 10 | 2.0% Fe₂O₃ | 183 |

In order to gather additional data to support the observations made by the AFM affinity method, additional experiments were performed with a solution affinity test.

A bovine tooth was cut in half lengthwise with a Dremel 400|XPR equipped with a Flex Shaft and a #545 diamond wheel. The enamel was then ground off the surface of the tooth to expose the dentin with the same Dremel equipped with a #8193 aluminum oxide grinding stone. Once the dentin was exposed, the surface was smoothed by sanding with 200 and 400 grit sandpaper (McMaster-Carr Silicon Carbide sandpaper). The dentin was then polished with a 50% silica flour (US Silica) slurry. It was then rinsed with deionized water and was polished again with a 50% slurry of calcium carbonate (HUBERCAL® 950). After polishing, the tooth was sonicated for 2 minutes in a 0.5 M HCl solution and was rinsed with deionized water.

Teflon tape was cut in half lengthwise and was wrapped around the middle of the polished tooth creating, two exposed and one unexposed sections. The unexposed section was used as a control for comparison during the test. The tooth was gripped along its side with tweezers and was submerged in an aqueous slurry of silica (10.0 g silica, 150-mL beaker, 90 mL deionized H₂O), that was stirred at a setting of 5 on a Thomas Magnematic model 15 stirplate for four minutes. During this time, the tooth was moved through the slurry with the dentin oriented into oncoming flow of silica particles. After the mixing time, the tooth was removed from the solution and was rinsed with deionized water for two seconds with a 500-mL squirt bottle. After the rinsing step, the sectioned tooth was allowed to dry at room temperature. Once dry, the Teflon tape was carefully removed and the tooth was analyzed by SEM.

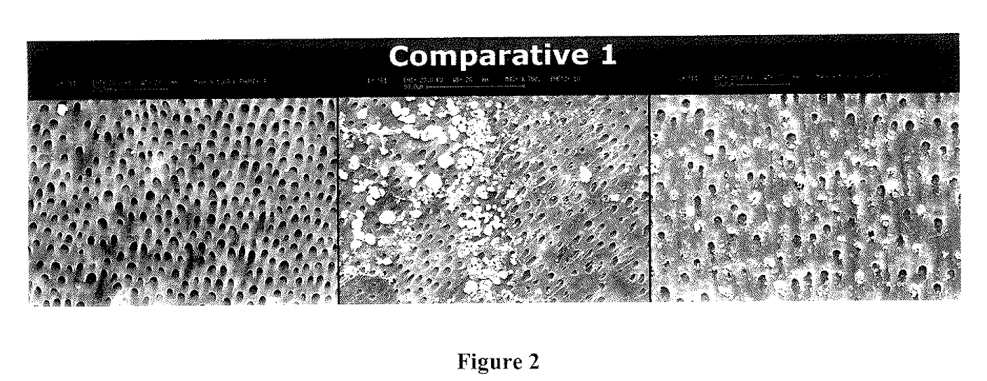

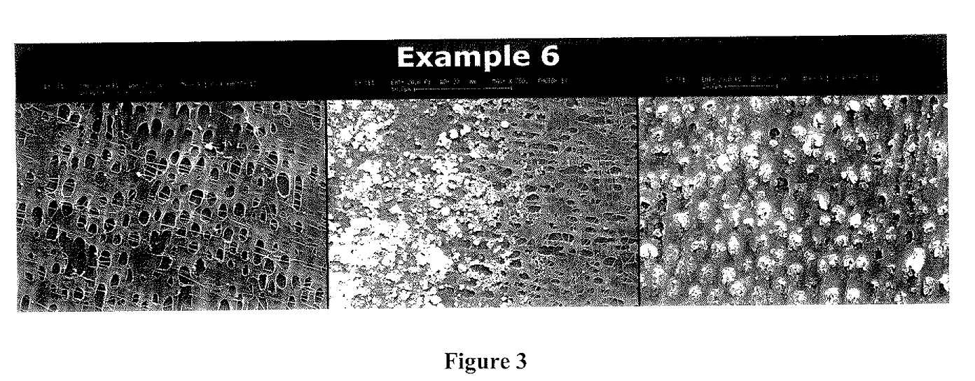

For the solution affinity test, both Comparative 1 and the Example 6 sample were evaluated. The tests were repeated several times, with representative results shown in FIGS. 2 (Comparative 1) and 3 (Example 6 silica). In FIGS. 2 and 3, the left side of the image shows the unexposed section of the tooth; the center of the image shows the boundary between the unexposed with exposed section; and the right side of the image shows the exposed section of the tooth.

It was observed that the tooth treated with the Example 6 silica (with 2% aluminum adduct) has greater surface coverage than Comparative 1 made with no adduct. These results of the solution affinity test agree with the observations of the AFM affinity test method in that the silica with adduct should be more efficient at occluding tubules in mammalian teeth.

### Dentifrice Production and Analysis of Tooth Surface Contact Therewith

Selected inventive examples from above were then incorporated into dentifrice formulations in accordance with the information provided in Table 10, below.

TABLE 10
Formulation Data for Dentifrice Samples

| | BATCH FORMULATION | | | | | |
|---|---|---|---|---|---|---|
| COMPONENT | 1 | 2 | 3 | 4 | 5 | 6 |
| Glycerine, | 11.600 | 11.600 | 11.600 | 11.600 | 11.600 | 11.600 |
| 99.5% | | | | | | |
| Sorbitol, 70.0% | 41.320 | 41.320 | 41.320 | 41.320 | 41.320 | 41.320 |
| Deionized | 18.097 | 18.097 | 18.097 | 18.097 | 18.097 | 18.097 |
| Water | | | | | | |
| Carbowax 600 | 3.000 | 3.000 | 3.000 | 3.000 | 3.000 | 3.000 |
| Cekol 2000 | 0.600 | 0.600 | 0.600 | 0.600 | 0.600 | 0.600 |
| Tetrasodium | 0.440 | 0.440 | 0.440 | 0.440 | 0.440 | 0.440 |
| Pyrophosphate | | | | | | |
| Sodium | 0.200 | 0.200 | 0.200 | 0.200 | 0.200 | 0.200 |
| Saccharin | | | | | | |
| Sodium | 0.243 | 0.243 | 0.243 | 0.243 | 0.243 | 0.243 |
| Fluoride | | | | | | |
| Thickener | | | | | | |
| Zeodent 165* | 5.000 | 5.000 | 5.000 | | | 5.000 |
| Comparative 4 | | | | 5.000 | | |
| [Zeothix 177*] | | | | | | |
| Comparative 5 | | | | | 5.000 | |
| [Zeothix 265*] | | | | | | |
| Abrasive | | | | | | |
| Zeodent 113* | 17.000 | 12.000 | 12.000 | 17.000 | 17.000 | 12.000 |
| Comparative 1 | | 5.000 | | | | |
| Example 6 | | | 5.000 | | | |
| Comparative 2 | | | | | | 5.000 |
| Sodium Lauryl | 1.500 | 1.500 | 1.500 | 1.500 | 1.500 | 1.500 |
| Sulfate | | | | | | |
| Flavor | 1.000 | 1.000 | 1.000 | 1.000 | 1.000 | 1.000 |
| Total | 100.000 | 100.000 | 100.000 | 100.000 | 100.000 | 100.000 |

*ZEODENT ® and ZEOTHIX ® products are precipitated silica materials available from J. M. Huber Corporation

These formulations were then analyzed for thickening capability to determine if the small particle-size inventive materials provided effective viscosity modification of the target dentifrice formulation when included with a precipitated silica abrasive (Zeodent 113). The viscosity measurements were tabulated and are presented in Table 10, below. Such results show that no deficiencies in thickening capabilities exist when utilizing this inventive metal adduct-treated precipitated silica material (not all formulations were measured for viscosity at every time interval, as noted below).

TABLE 11
Viscosity Data for Dentifrice Samples (×1000 cP)

| | Sample | | | | | |
|---|---|---|---|---|---|---|
| | Formulation | | | | | |
| | Number | | | | | |
| Time | 1 | 2 | 3 | 4 | 5 | 6 |
| (Days) | Control | Comparative 1 | Example 6 | Comparative 4 | Comparative 5 | Comparative 2 |
| 1 | — | — | — | — | — | 239 |
| 3 | 252 | 272 | 283 | 306 | 276 | — |
| 7 | 275 | 290 | 314 | 346 | 300 | 305 |
| 21 | 353 | 353 | 386 | 388 | 380 | — |
| 42 | 406 | 388 | 468 | 510 | 419 | — |

To determine the effect particle size has on the ability of the inventive precipitated silica materials to occlude target dentinal tubules, as well as the ability of such materials to transfer from a dentifrice formulation to a target tooth surface (and ultimately within the tubules therein), further testing was undertaken, specifically in terms of the same solution affinity test described above, but for the results after 1 minute of brushing with 2 grams of dentifrice (from the above Table 9) applied to the subject treated bovine teeth (hereinafter the “dentifrice affinity test”). As for the same solution affinity test outlined above, a half-inch piece of TEFLON® (DuPont) tape was cut in half lengthwise and wrapped around the middle of the tooth, effectively creating three different sections, two exposed and one unexposed. The unexposed section was the internal standard during the test.

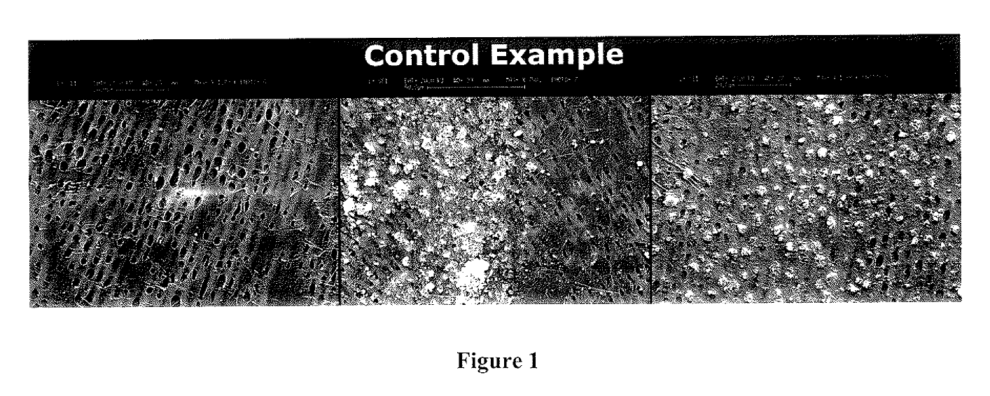

For this dentifrice affinity test, five samples were evaluated: one Control sample, Comparative 1, Example 6, Comparative 4, Comparative 5. FIGS. 1-5 show the results from the dentifrice affinity test. The tooth sections were brushed (Oral-B, soft-bristled, regular head toothbrush) with the requisite dentifrice for 1 minute. After brushing, the tooth was rinsed with deionized water until there was no visible residue left on the tooth (approximately 10 seconds).

### DETAILED DESCRIPTION OF THE DRAWINGS

For each of the provided FIGS. 1-6, the images are arranged as follows: 1) the left side of the image shows the image of the unexposed section of the tooth, 2) the center of the image shows the image of the boundary between the unexposed and exposed sections, and 3) the right side of the image shows the image of the exposed section of the tooth.

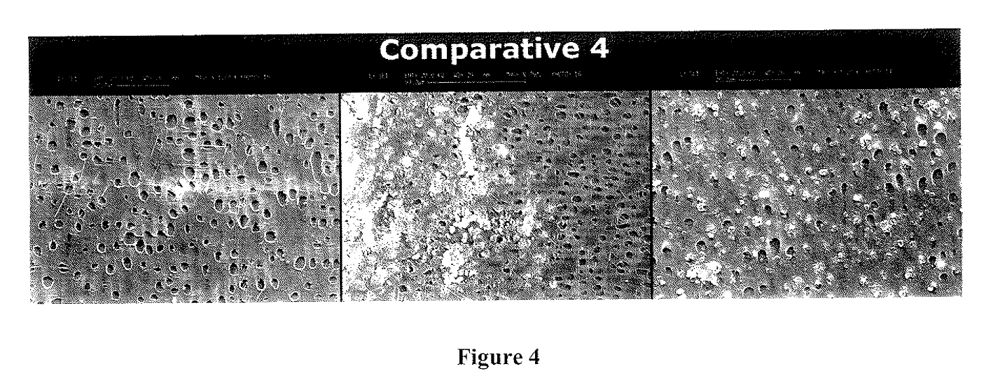

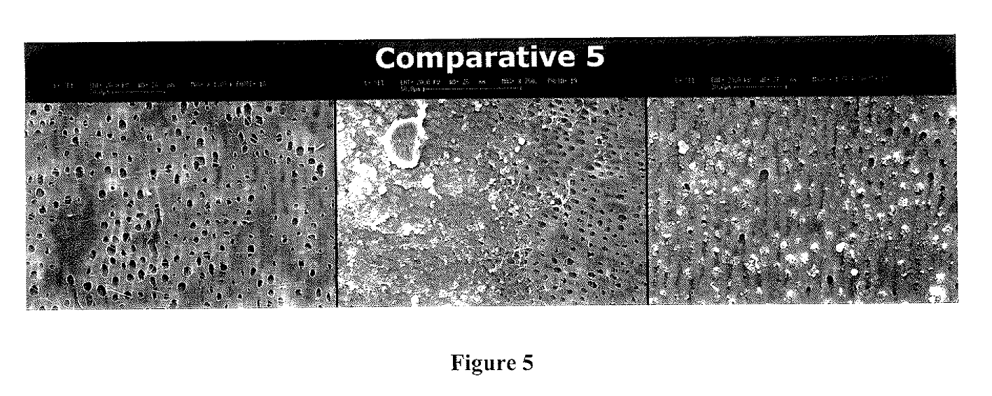

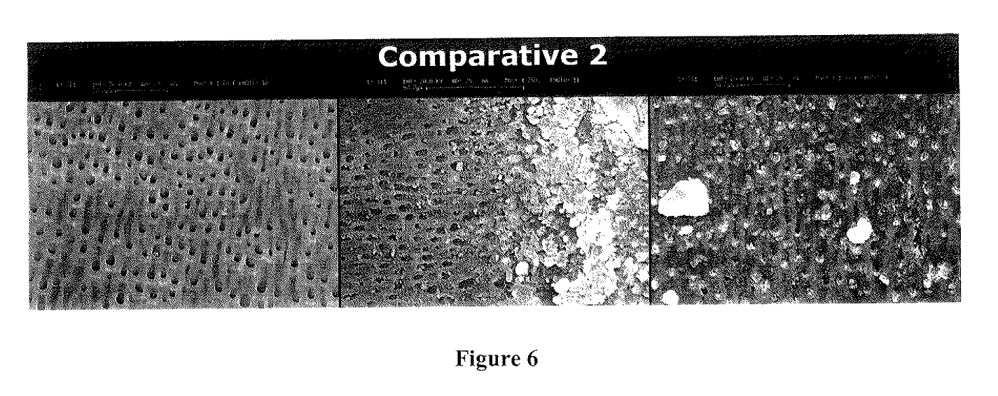

From the images shown in these FIGS. 1-6, it can be seen that Example 6 (FIG. 3) visually shows that the inventive silica materials therein exhibit a greater affinity and coverage of the dentin surface, as well as over and within the tubules, compared to the Control and Comparatives. This data correlates well with the data obtained using the AFM in that the doped silica should be better suited at occluding tubules in teeth and also with the solution affinity test which exemplifies the same phenomenon. FIGS. 1 and 2 show little to no coverage of this sort. FIGS. 4 and 5 show a larger degree of coverage than FIGS. 1 and 2. Furthermore, the smaller particle size examples (in FIGS. 3-5) provide greater coverage, clearly, than that provided in FIG. 6 (larger milled silica particles treated with metal adduct). Even with the metal adduct present thereon, the size of the particles are too large to provide effective coverage within the subject tubules; only adhesion to the dentin surface is observed to any degree. In FIG. 6, some fines present with the large particle example do make their way into some of the tubules; however, the majority of the particles are too large to have any beneficial tubule-filling effect. FIG. 6, in particular, shows with proper particle size distribution that a result can be attained that is conducive to a large amount of silica material to adhere to, build, and fill the target tubules for sensitivity reduction to occur.

While the invention has been described in detail with respect to specific embodiments thereof, it will be appreciated that those skilled in the art, upon attaining an understanding of the foregoing, may readily conceive of alterations to, variations of, and equivalents to these embodiments. Accordingly, the scope of the invention should be assessed as that of the appended claims and any equivalents thereof.

We claim:

1. A precipitated silica material having a mean particle size of 1 to 5 microns and having an adduct present on at least a portion of its surface to form an adduct-treated precipitated silica material, wherein the adduct-treated precipitated silica material exhibits a zeta potential reduction greater than 10% when compared to a precipitated silica material of the same structure on which no adduct is present.

2. The precipitated silica material of claim 1 wherein the adduct is a metal element.

3. The precipitated silica material of claim 2 wherein the metal element is selected from the transition metals or post-transition metals.

4. The precipitated silica material of claim 3 wherein the metal element is selected from the group consisting of aluminum, zinc, tin, strontium, iron, copper and mixtures thereof.

5. The precipitated silica material of claim 1 wherein the adduct-treated precipitated silica material exhibits a zeta potential reduction greater than 15% when compared to a precipitated silica material of the same structure on which no adduct is present.

6. The precipitated silica material of claim 1 wherein the adduct-treated precipitated silica material exhibits a zeta potential reduction greater than 20% when compared to a precipitated silica material of the same structure on which no adduct is present.

7. The precipitated silica material of claim 1 wherein the adduct-treated precipitated silica material exhibits a zeta potential reduction greater than 25% when compared to a precipitated silica material of the same structure on which no adduct is present.

8. A dentifrice comprising the adduct-treated precipitated silica material as defined in claim 1 and at least one other component selected from the group consisting of at least one abrasive other than the adduct-treated precipitated silica material, at least one thickening agent other than the adduct-treated precipitated silica material, at least one solvent, at least one preservative, and at least one surfactant, wherein the adduct-treated precipitated silica material is present as an abrasive agent, thickening agent, or both, within the dentifrice.

9. A dentifrice comprising the adduct-treated precipitated silica material as defined in claim 5 and at least one other component selected from the group consisting of at least one abrasive other than the adduct-treated precipitated silica material, at least one thickening agent other than the adduct-treated precipitated silica material, at least one solvent, at least one preservative, and at least one surfactant, wherein the adduct-treated precipitated silica material is present as an abrasive agent, thickening agent, or both, within the dentifrice.

10. A dentifrice comprising the adduct-treated precipitated silica material as defined in claim 6 and at least one other component selected from the group consisting of at least one abrasive other than the adduct-treated precipitated silica material, at least one thickening agent other than the adduct-treated precipitated silica material, at least one solvent, at least one preservative, and at least one surfactant, wherein the adduct-treated precipitated silica material is present as an abrasive agent, thickening agent, or both, within the dentifrice.

11. A dentifrice comprising the adduct-treated precipitated silica material as defined in claim 7 and at least one other component selected from the group consisting of at least one abrasive other than the adduct-treated precipitated silica material, at least one thickening agent other than the adduct-treated precipitated silica material, at least one solvent, at least one preservative, and at least one surfactant, wherein the adduct-treated precipitated silica material is present as an abrasive agent, thickening agent, or both, within the dentifrice.

12. A method of treating a mammalian tooth comprising the steps of
a) providing a dentifrice comprising a precipitated silica material having a mean particle size of 1 to 5 microns and having an adduct present on at least a portion of its surface to form an adduct-treated precipitated silica material that exhibits a zeta potential reduction greater than 10% when compared to a precipitated silica material of the same structure on which no adduct is present;
b) applying the dentifrice to a mammalian tooth; and
c) brushing the dentifrice-applied tooth of step “b”.

13. The method of claim 12 wherein the dentifrice of step “a” further comprises at least one other component selected from the group consisting of at least one abrasive other than the adduct-treated precipitated silica material, at least one thickening agent other than the adduct-treated precipitated silica material, at least one solvent, at least one preservative, and at least one surfactant, wherein the adduct-treated precipitated silica material is present as an abrasive agent, thickening agent, or both, within the dentifrice.

14. The method of claim 12 wherein the adduct-treated precipitated silica material of step “a” exhibits a zeta potential reduction greater than 15% when compared to a precipitated silica material of the same structure on which no adduct is present.

15. The method of claim 12 wherein the adduct-treated precipitated silica material of step “a” exhibits a zeta potential reduction greater than 20% when compared to a precipitated silica material of the same structure on which no adduct is present.

16. The method of claim 12 wherein the adduct-treated precipitated silica material of step “a” exhibits a zeta potential reduction greater than 25% when compared to a precipitated silica material of the same structure on which no adduct is present.

17. The method of claim 13 wherein the adduct-treated precipitated silica material of step “a” exhibits a zeta potential reduction greater than 15% when compared to a precipitated silica material of the same structure on which no adduct is present.

18. The method of claim 17 wherein the adduct-treated precipitated silica material of step “a” exhibits a zeta potential reduction greater than 20% when compared to a precipitated silica material of the same structure on which no adduct is present.

19. The method of claim 17 wherein the adduct-treated precipitated silica material of step “a” exhibits a zeta potential reduction greater than 25% when compared to a precipitated silica material of the same structure on which no adduct is present.
